(19) United States
(12) Patent Application Publication
(10) Pub. No.: US 2025/0160822 A1
(43) Pub. Date: May 22, 2025

(54) SINGLE BARRIER FOLDER SUTURE PACKAGE

(71) Applicant: Ethicon, Inc., Raritan, NJ (US)

(72) Inventors: Raouf SALEH, Staten Island, NY (US); Jack LUMLEY, Johnstown, PA (US); Mohit AGARWAL, Aurangabad (IN)

(21) Appl. No.: 18/838,104

(22) PCT Filed: Feb. 9, 2023
(86) PCT No.: PCT/IB2023/051179

§ 371 Date: Aug. 13, 2024

(30) Foreign Application Priority Data
Feb. 14, 2022 (IN) 202211007688

(51) Int. Cl. A61B 17/06

(52) U.S. Cl. CPC A61B 17/06133; A61B 2017/06147

(57) ABSTRACT

Various embodiments of the present invention are directed towards a single barrier folder to carry a suture and a needle therein. The single barrier folder comprises a plurality of openings configured to allow placement of a suture and form a suture winding and a first flap comprising a needle parking mechanism. The needle parking mechanism comprises a first portion, a notch, and a cutout. The cutout and the notch are configured to hold the needle and the first portion is foldable and configured to fold in locking engagement with the notch. The single barrier folder also comprises a second flap and a third flap on opposite sides of the plurality of openings and a slit configured to receive an end portion of a fourth flap to lock the single barrier folder. The present invention also provides method of placing and folding the flaps to pack needle and suture inside the single barrier folder.

### TECHNICAL FIELD OF INVENTION

The present invention generally relates to packaging of items. More particularly, the invention relates to a system and a method providing packaging of sutures and needles in a single barrier folder.

### BACKGROUND OF THE INVENTION

Distribution of medical devices such as needles, sutures from factories or development centers is done by packing them in bulk, in different cartons. In each carton, a unit of suture as well as needle is packed in a package called as single barrier folder (SBF). In the single barrier folder, the sutures and needles are wrapped and transported in cartons.

The needles and sutures are kept in the SBF where the objective is to keep them sterile. However, during transit and transportation of the packages/SBF there may be jerks and movements therein where it is possible that the needles and sutures inside the SBF may move and slip or pierce out of the package. In this case, the suture and needles may not remain sterile and may be unfit for medical use.

Moreover, existing design of the SBF is common for different types of needles such as curved needles as well as straight needles. Therefore, the SBF is not suitable to hold every type of needles. Further, the current SBF designs have sharp edges on flaps where it is highly possible that the SBF may get ruptured if the needle inside comes in contact with the edges due to any external force.

Additionally, the locking mechanism in an SBF is simple where only linear grooves and slots are provided to lock and pack it, leaving a high possibility that it may open up during transit and the needles and sutures may be exposed to outside environment and are likely to get contaminated.

In view of the afore-mentioned problems in the existing solutions, there is a need of a modified SBF which does not have sharp edges and is easy to fold. There is a need in the art to have proper needle and suture placement mechanisms such that the sutures and needles inside the packages do not move during transit. There is a need in the art to provide extended and enhanced flap structures in SBF for holding the sutures. There is also a need to provide a robust locking mechanism for the needles and sutures inside SBF such that accidental rupturing of the SBF is greatly reduced and there is better alignment of locking flap. In order to solve the problems in the existing solutions, a modified SBF and a method are disclosed.

### SUMMARY OF THE INVENTION

The following presents a simplified summary of the invention in order to provide a basic understanding of some aspects of the invention. This summary is not an extensive overview of the present invention. It is not intended to identify the key/critical elements of the invention or to delineate the scope of the invention. Its sole purpose is to present some concept of the invention in a simplified form as a prelude to a more detailed description of the invention presented later.

Various embodiments of the invention describe a single barrier folder for holding a suture and a needle. The single barrier folder comprises a plurality of openings configured to allow placement of a suture and form a suture winding and a first flap comprising a needle parking mechanism. The needle parking mechanism comprises a first portion, a notch, and a cutout. The cutout and the notch are used to hold the needle and the first portion is foldable and configured to fold in locking engagement with the notch. The single barrier folder also comprises a second flap and a third flap on opposite sides of the plurality of openings. Moreover, a slit is configured to receive an end portion of a fourth flap to lock the single barrier folder.

In an embodiment of the invention, the plurality of openings allows external rods to be inserted through the plurality of openings for forming the suture winding.

In another embodiment of the invention, the plurality of openings are provided in a central zone of the single barrier folder.

In another embodiment of the invention, the notch is present on a foldable perforated edge between the first flap and the central zone.

In another embodiment of the invention, the first flap, second flap, and the third flap have rounded edges.

In yet another embodiment of the invention, the surface area of the third flap is larger than surface area of the second flap.

In an embodiment of the invention, the cutout is trapezoidal in shape and the notch is V-shaped.

In yet another embodiment of the invention, the slit is provided along with the plurality of openings in the central zone and the slit is curved.

In still another embodiment of the invention, the fourth flap comprises a protrusion which allows the end portion of the fourth flap to lock into the slit.

In a different embodiment of the invention, the fourth flap further comprises linear perforations to tear off a portion of fourth flap.

In another embodiment of the invention, the slit is provided on a back face of the central zone and the fourth flap is folded covering the suture and the needle to lock into the slit.

In an embodiment of the invention, the notch and cutout are configured to hold the needle which is curved.

In a different embodiment of the invention, the first flap and fourth flap are provided on opposite sides of the plurality of openings and the first flap and fourth flap are perpendicularly located with respect to the second flap and the third flap.

In various embodiments of the invention, a method of locking a single barrier folder is disclosed. The method comprises placing a suture and forming a suture winding using a plurality of openings. The method further comprises folding a first flap where the first flap comprises a needle parking mechanism. The needle parking mechanism comprises a first portion, a notch, and a cutout, wherein the cutout and the notch are used to hold the needle and the first portion is foldable and configured to fold in locking engagement with the notch. The method also comprises folding a second flap and subsequently a third flap present on opposite sides of the plurality of openings and folding a fourth flap. The fourth flap comprises an end portion which is configured to lock the single barrier folder using a slit.

In an embodiment of the invention, the plurality of openings allows external rods to be inserted through the plurality of openings for forming the suture winding.

In another embodiment of the invention, the plurality of openings are provided in a central zone of the single barrier folder.

In another embodiment of the invention, the notch is present on a foldable perforated edge between the first flap and the central zone.

In another embodiment of the invention, the first flap, second flap, and the third flap have rounded edges.

In yet another embodiment of the invention, the surface area of the third flap is larger than surface area of the second flap.

In an embodiment of the invention, the cutout is trapezoidal in shape and the notch is V-shaped.

In yet another embodiment of the invention, the slit is provided along with the plurality of openings in the central zone and the slit is curved.

In still another embodiment of the invention, the fourth flap comprises a protrusion which allows the end portion of the fourth flap to lock into the slit. In a different embodiment of the invention, the fourth flap further comprises linear perforations to tear off a portion of fourth flap.

In another embodiment of the invention, the slit is provided on a back face of the central zone and the fourth flap is folded covering the suture and the needle to lock into the slit.

In an embodiment of the invention, the notch and cutout are configured to hold the needle which is curved.

In a different embodiment of the invention, the first flap and fourth flap are provided on opposite sides of the plurality of openings and the first flap and fourth flap are perpendicularly located with respect to the second flap and the third flap.

Various embodiments of the invention describes a single barrier folder for holding a suture and a needle. The single barrier folder comprises a plurality of openings configured to allow placement of a suture and form a suture winding. The single barrier folder comprises a first flap comprising a needle parking mechanism. The needle parking mechanism comprises a first portion, a notch, a first cutout and a second cutout. The first cutout and the second cutout are used to hold the needle and the first portion is foldable and configured to fold in locking engagement with the notch. The single barrier folder comprises a second flap and a third flap on each side of the plurality of openings. A slit is configured to receive an end portion of a fourth flap to lock the single barrier folder and the slit. The slit is curved.

Various embodiments of the invention describe a single barrier folder comprising a plurality of openings configured to allow placement of a suture and form a suture winding. The single barrier folder comprises a first flap comprising a foam section for holding the needle. A second flap and a third flap are present on opposite sides of the plurality of openings. A slit is configured to receive an end portion of a fourth flap to lock the single barrier folder and the slit. The slit is curved.

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

Other aspects, advantages, and salient features of the invention will become apparent to those skilled in the art from the following detailed description, which taken in conjunction with the annexed drawings, discloses exemplary embodiments of the invention.

### BRIEF DESCRIPTION OF THE DRAWINGS

Some of the objects of the invention have been set forth above. These and other objects, features, aspects and advantages of the present invention will become better understood with regard to the following description, appended claims and accompanying drawings where: FIG. 1 depicts an architecture of single barrier folder for placing curved needle and suture according to an exemplary embodiment of the invention. FIG. 2 depicts an architecture of a single barrier placing straight needle and suture according to an exemplary embodiment of the invention.

FIG. 3 depicts an architecture of a single barrier folder for placing needle and suture according to another exemplary embodiment of the invention.

FIG. 4 depicts a flowchart use the single barrier folder for needle and suture according to another exemplary embodiment of the invention.

Corresponding reference numerals indicate corresponding parts throughout the drawings.

### DETAILED DESCRIPTION OF THE INVENTION

The following detailed description should be read with reference to the drawings in which similar elements in different drawings are numbered the same. The drawings, which are not necessarily to scale, depict illustrative embodiments and are not intended to limit the scope of the invention. Persons skilled in the art will recognize that many of the examples provided have suitable alternatives that may be utilized interchangeably and the dimensions of different features may be modified.

Described herein is the inventive configuration of and a single barrier folder provided with locking mechanism and other features which allow safe transit of the components placed inside the single barrier folder. The single barrier folder comprises a plurality of openings configured to allow placement of a suture and form a suture winding therein. The single barrier folder comprises various flaps including features which are configured to provide safe placement of needle and suture during transit. The single barrier folder uses a parking mechanism for needle as well as placement of suture such that the relative movement of the suture and needle inside the single barrier folder is minimized. The single barrier folder also comprises a locking mechanism to hold the suture and needle.

As used herein, the single barrier folder comprises a cut out structure for packing with paper of higher thickness. The single barrier folder comprises various notches, crease, and perforations for providing packaging structure.

As used herein, the definition of various terms such as notches, slit, perforation, protrusion, crease, openings and the like are known to a person skilled in the art.

FIG. 1 discloses an exemplary architecture (100) of a single barrier folder according to an exemplary embodiment of the invention. As disclosed in FIG. 1, the single barrier folder is divided into various sections including a first flap (102), a second flap (104), a third flap (106), and a fourth flap (108). The single barrier folder 100 also comprises a central zone (110) comprising a plurality of openings (112a, 112b, 112c, 112d) and a slit (114).

As shown in FIG. 1, the plurality of openings (112a, 112b, 112c, 112d) is configured to allow external features such as rods of suitable dimensions to be inserted through the plurality of openings. The external features such as rods may be affixed to any structure or platform which may represent a stable structure to allow fixation of the single barrier folder to assist in packing of suture and needle inside the single barrier folder. The external rods are only a kind of structures for placing the single barrier folder known to a person skilled in the art. The plurality of openings (112a, 112b, 112c, 112d) in the single barrier folder are defined to allow effective placement of the single barrier folder. On insertion of the rods through the openings and placing the single barrier folder, a suture of predetermined length is taken and woven around the rods. The suture is woven in a specified structure around the rods in the form of winding such that a suture winding is formed. The suture winding may be taken off the rods for placement into the single barrier folder centrally over the central zone and over the plurality of openings. The plurality of openings may be located such that two of the openings are located near the left edge of the single barrier folder and the two openings may be located near the right edge of the single barrier folder.

The single barrier folder 100 comprises a first flap (102) with various features to allow placement of a needle. The first flap (102) comprises a first portion (116) which is cut along the edges such that it is foldable vertically. The first portion (116) may be moved and lifted and get folded along uncut edge of the first portion (116). The first flap (102) comprises a cutout (118) which may be used to place a portion of needle therein horizontally. The cutout (118) may have a trapezoidal cut structure such that the needle is placed effectively and does not jump out due to any jerk. The first flap also comprises a notch (120) on an edge between the central zone (110) and the first flap (102). The notch (120) may be a V-shaped notch which allows placement of another portion of the needle. The notch (120) and the cutout (118) are configured to hold the needle. In an embodiment of the invention, the needle placed in the cutout (118) and the notch (120) is a curved needle.

After placing the suture using the plurality of openings (112a, 112b, 112c, 112d), the first flap (102) may be folded over the central zone (110) along a perforated edge on which the notch (120) is present. The needle is then placed inside the cutout (118) and the notch (120) as discussed above. At least a portion of the suture placed over the central zone may be lockingly engaged with the cutout (118). The first portion (116) is then folded upwardly to lockingly engage with the notch (120) thus securing the needle therein.

In another embodiment of the invention, the first portion (116) may also be folded such that the locking engagement is provided with both the notch (120) as well as the cutout (118). This provides further benefits of improved locking and safe transit of the needle and suture.

The first flap (102) may have rounded corners to eliminate sharp edges of fully assembled folder to avoid any rupture in the single barrier folder.

FIG. 1 further illustrates the second flap (104) having rounded corners. The third flap is extended and the surface area of the third flap (106) is larger than the second flap (104). The surface area of the second flap (106) is larger to avoid movement of the suture such that it does not come out of the single barrier folder during transit. The third flap (106) also has rounded corners to eliminate sharp edge to avoid any rupture of the single barrier folder.

FIG. 1 further illustrates a fourth flap (108) divided into at least three sections. Section (108a) is separated from section (108b) with perforations (shown with dashed line). By using perforations, the sections (108b) and (108c) can be separated from section (108a). The sections (108b) and (108c) are separated from each other using a crease which can be folded as the fourth flap is folded over the central zone (110). It is to be noted that sections (108b) and (108c) can be folded along boundary between the fourth flap (108) and the central zone (110).

After placing of suture and folding of the first flap (102) and subsequent placement of needle and locking engagement of the notch (120) and the first portion (116), the second flap (104) is folded over the first flap (102) and the central zone (110). Thereafter, the third flap (106) is folded over the first flap (102) and the central zone (110). The second flap (104) and the third flap (106) are folded such that both of them are placed adjacently over the central zone (110) and the first flap (102). Thereafter, the fourth flap (108) is folded over the second flap (104) and third flap (106). The section (108a) may be teared off from the sections (108b) and (108c) as discussed above during folding of the fourth flap (108).

It may be noted that the slit (114) present in the central zone (110) is configured to lockingly engage the fourth flap (108) and in particular, section (108c) of the fourth flap (108) with the slit (114). The slit (114) may be curved as shown in FIG. 1 and is configured to get lockingly engage with the section (108c) on back side of the central zone (110). In particular, when the fourth flap (108) is folded over central zone (110) and the second and third flaps specifically, section (108b) folds over the third flap (106). Further, the section (108c) is folded over the back side of the central zone (110) after passing over the third flap (106). The section (108c) may have a protrusion (108c1) within the fourth flap (108) and the protrusion (108c1) includes an end portion (108c2) having a rounded edge which may be inserted in the slit (114) on the back side of the central zone to pack the single barrier folder (100). The end portion (108c2) of the protrusion (108c1) may sit inside the slit (114) in locking engagement. The section (108a) may lie over the folded second flap (104). In particular, the fourth flap (108) is folded covering the suture and the needle which lie under the second flap (104) and the third flap (106). The curved slit (114) allows better locking engagement between the fourth flap (108) and the slit (114).

The placement of the different flap discussed above is such that the first flap (102) and fourth flap (108) are provided on opposite sides of the plurality of openings (112a, 112b, 112c, 112d) and the first flap (102) and the fourth flap (108) are perpendicularly located with respect to the second flap (104) and the third flap (106).

Turning now to FIG. 2, which discloses an exemplary architecture (200) of a single barrier folder according to an exemplary embodiment of the invention. It is noted that single barrier folder of FIG. 2 has provisions of holding a straight needle as compared to the features of single barrier folder in FIG. 1. As disclosed in FIG. 2, the single barrier folder is divided into various sections including a first flap (202), a second flap (204), a third flap (206), and a fourth flap (208). The single barrier folder also comprises a central zone (210) comprising a plurality of openings (212a, 212b, 212c, 212d) and a slit (214).

As shown in FIG. 2, the plurality of openings (212a, 212b, 212c, 212d) is configured to allow external features such as rods of suitable dimensions to be inserted through the plurality of openings The external features such as rods may be affixed to any structure or platform which may represent a stable structure to allow fixation of the single barrier folder to assist in packing of suture and needle inside the single barrier folder. The external rods are only a kind of structures for placing the single barrier folder known to person skilled in the art. The plurality of openings (212a, 212b, 212c, 212d) in the single barrier folder are defined to allow effective placement of the single barrier folder. On insertion of the rods through the openings and placing the single barrier folder, a suture of predetermined length is taken and woven around the rods. The suture is woven in a specified structure around the rods in the form of winding such that a suture winding is formed. The suture winding may be taken off the rods for placement into the single barrier folder centrally over the central zone and over the plurality of openings. The plurality of openings (212a, 212b, 212c, 212d) may be located such that two of the openings are located near the left edge of the single barrier folder and the two openings may be located near the right edge of the single barrier folder (200).

The single barrier folder (200) comprises a first flap (202) with various features to allow placement of a needle. The first flap (202) comprises a first portion (216) which is cut along the edges such that it is foldable vertically. The first portion (216) may be moved and lifted and get folded along uncut edge of the first portion (216). The first flap (202) comprises a first cutout (218) which may be used to place a portion of needle therein horizontally. The first cutout (218) may have a trapezoidal cut structure such that the needle is placed effectively and does not jump out due to any jerk. The first flap (202) also comprises a notch (220) on an edge between the central zone (110) and the first flap (202). The first flap (202) also comprises a second cut out (222) placed in line with the first cutout (218) horizontally to hold the needle. The first cutout (218) and second cutout (222) are configured to hold the needle which is straight. The profile of the second cut out (222) may be gaussian having a rounded cut at the top followed by a streamline cut in vertical direction. The profile allows the needle to be held with lesser chances of jumping out. The notch (120) may be a V-shaped notch and is configured to lockingly engaged with the first portion (216).

After placing the suture using the plurality of openings (212a, 212b, 212c, 212d), the first flap (202) may be folded over the central zone (210) along a perforated edge on which the notch (220) is present. The needle is then placed inside the first cutout (218) and the second cutout (222) as discussed above. At least a portion of the suture placed over the central zone may be lockingly engaged with the first cutout (218). The first portion (216) is then folded upwardly to lockingly engage with the notch (220) thus securing the needle therein.

In another embodiment of the invention, the first portion (216) may also be folded such that the locking engagement is provided with both the notch (220) as well as the first cutout (218). This, provides further benefits of improved locking and safe transit of the needle and suture.

The first flap (202) may have rounded corners to eliminate sharp edges of fully assembled folder to avoid any rupture in the single barrier folder.

FIG. 2 further illustrates the second flap (204) having rounded corners. The third flap (206) is extended and the surface area of the third flap (206) is larger than the second flap (204). The surface area of the second flap (206) is larger to avoid movement of the suture such that it does not come out of the single barrier folder during transit. The third flap (206) also has rounded corners to eliminate sharp edge to avoid any rupture of the single barrier folder.

FIG. 2 further illustrates a fourth flap (208) divided into at least three sections. Section (208a) is separated from section (208b) with perforations (shown with dashed line). By using perforations, the sections (208b) and (208c) can be separated from section (208a). The sections (208b) and (208c) are separated from each other using a crease which can be folded as the fourth flap is folded over the central zone (210). It is to be noted that sections (208b) and (208c) can be folded along boundary between the fourth flap (208) and the central zone (210).

After placing of suture and folding of the first flap (202) and subsequent placement of needle and locking engagement of the notch (220) and the first portion (216), the second flap (204) is folded over the first flap (202) and the central zone (210). Thereafter, the third flap (206) is folded over the first flap (202) and the central zone (210). The second flap (204) and the third flap (206) are folded such that both of them are placed adjacently over the central zone (210) and the first flap (202). Thereafter, the fourth flap (208) is folded over the second flap (204) and third flap (206). The section (208a) may be teared off from the sections (208b) and (208c) as discussed above during folding of the fourth flap (208).

It may be noted that the slit (214) present in the central zone (210) is configured to lockingly engage the fourth flap (208) and in particular, section (208c) of the fourth flap (208) with the slit (214). The slit (214) may be curved as shown in FIG. 2 and is configured to get lockingly engaged with the section (208c) on back side of the central zone (210). In particular, when the fourth flap (208) is folded over central zone (210) and the second and third flaps specifically, section (208b) folds over the third flap (206). Further, the section (208c) is folded over the back side of the central zone (210) after passing over the third flap (206). The section (208c) may have a protrusion (208c1) within the fourth flap (208) and the protrusion (208c1) includes an end portion (208c2) having a rounded edge which may be inserted in the slit (214) on the back side of the central zone to pack the single barrier folder (200). The end portion (208c2) of the protrusion (208c1) may sit inside the slit (214) in locking engagement. The section (208a) may lie over the folded second flap (204). In particular, the fourth flap (208) is folded covering the suture and the needle which lie under the second flap (204) and the third flap (206). The curved slit (214) allows better locking engagement between the fourth flap (208) and the slit (214).

The placement of the different flaps discussed above are such that the first flap (202) and fourth flap (208) are provided on opposite sides of the plurality of openings (212a, 212b, 212c, 212d) and the first flap (202) and the fourth flap (208) are perpendicularly located with respect to the second flap (204) and the third flap (206).

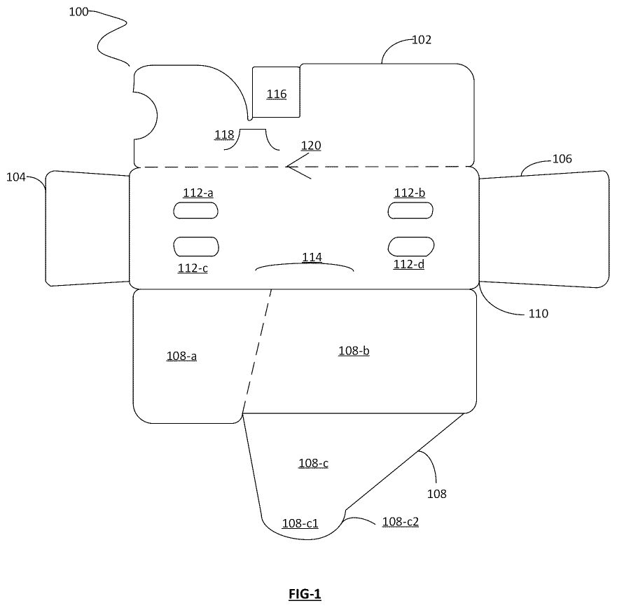

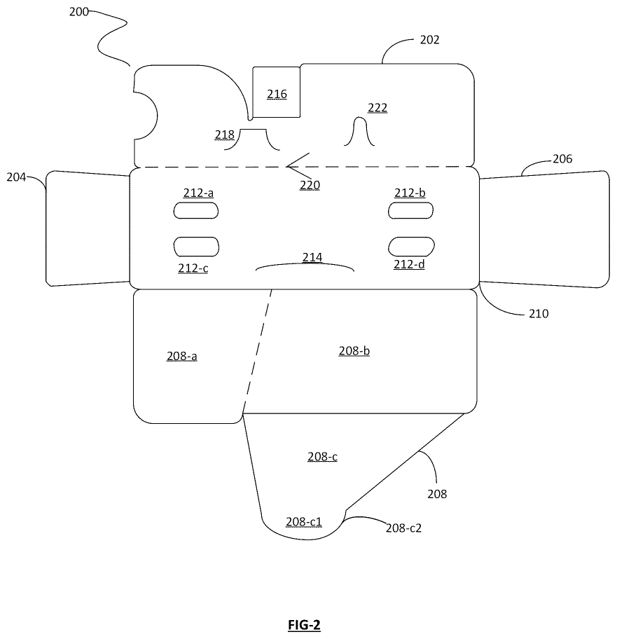

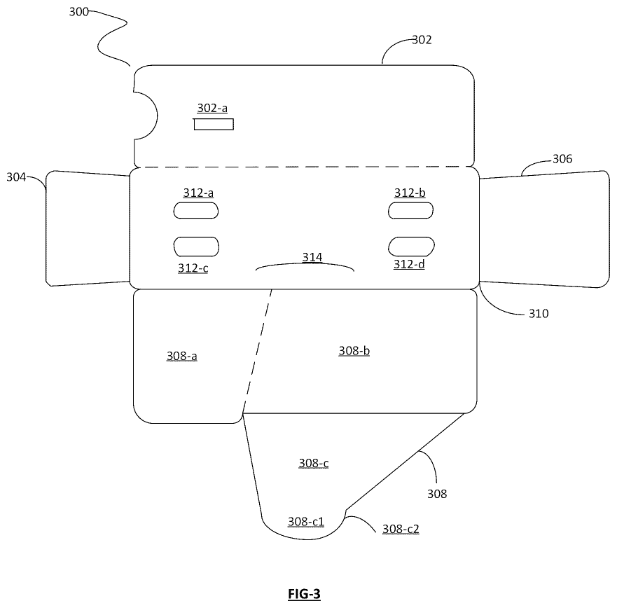

Turning now to FIG. 3, FIG. 3 discloses an exemplary architecture (300) of a single barrier folder according to an exemplary embodiment of the invention. It is noted that single barrier folder (300) of FIG. 3 has provision of holding a straight needle as well as curved needle as compared to the features of single barrier folder in FIG. 1 and FIG. 2. As disclosed in FIG. 3, the single barrier folder is divided into various sections including a first flap (302), a second flap (304), a third flap (306), and a fourth flap (308). The single barrier folder (300) also comprises a central zone (310) comprising a plurality of openings (312a, 312b, 312c, 312d) and a slit (314).

As shown in FIG. 3, the plurality of openings (312a, 312b, 312c, 312d) is configured to allow external features such as rods of suitable dimensions to be inserted through the plurality of openings The external features such as rods may be affixed to any structure or platform which may represent a stable structure to allow fixation of the single barrier folder (300) to assist in packing of suture and needle inside the single barrier folder. The external rods are only a kind of structures for placing the single barrier folder known to person skilled in the art. The plurality of openings (312a, 312b, 312c, 312d) in the single barrier folder are defined to allow effective placement of the single barrier folder. On insertion of the rods through the openings and placing the single barrier folder, a suture of predetermined length is taken and woven around the rods. The suture is woven in a specified structure around the rods in the form of winding such that a suture winding is formed. The suture winding may be taken off the rods for placement into the single barrier folder centrally over the central zone and over the plurality of openings. The plurality of openings (312a, 312b, 312c, 312d) may be located such that two of the openings are located near the left edge of the single barrier folder and the two openings may be located near the right edge of the single barrier folder.

The single barrier folder (300) comprises a first flap (302) with a foam section (302a) for placing a needle therein. The needle may be inserted in the foam section which acts as a holder for the needle. The needle may be a curved needle or a straight needle. It may be noted that the foam section (302a) may be positioned anywhere on the first flap. The position shown in FIG. 3 is exemplary.

After placing the suture using the plurality of openings (212a, 212b, 212c, 212d), the first flap (202) may be folded over the central zone (210) along a perforated edge along boundary of the first flap (302) and the central zone (310).

The first flap (202) may have rounded corners to eliminate sharp edges of fully assembled folder to avoid any rupture in the single barrier folder.

FIG. 3 further illustrates the second flap (304) having rounded corners. The third flap (306) is extended and the surface area of the third flap (306) is larger than the second flap (304). The surface area of the second flap (306) is larger to avoid movement of the suture such that it does not come out of the single barrier folder during transit. The third flap (306) also has rounded corners to eliminate sharp edge to avoid any rupture of the single barrier folder.

FIG. 3 further illustrates a fourth flap (308) divided into at least three sections. Section (308a) is separated from section (308b) with perforations (shown with dashed line). By using perforations, the sections (308b) and (308c) can be separated from section (308a). The sections (308b) and (308c) are separated from each other using a crease which can be folded as the fourth flap (308) over the central zone (310). It is to be noted that sections (308b) and (308c) can be folded along boundary between the fourth flap (308) and the central zone (310).

After placing of suture and the needle in the foam section (302a) and folding of the first flap (302), the second flap (304) is folded over the first flap (302) and the central zone (310). Thereafter, the third flap (306) is folded over the first flap (302) and the central zone (310). The second flap (304) and the third flap (306) are folded such that both of them are placed adjacently over the central zone (310) and the first flap (302). Thereafter, the fourth flap (308) is folded over the second flap (304) and the third flap (306). The section (308a) may be teared off from the sections (308b) and (308c) as discussed above during folding of the fourth flap (308).

It may be noted that the slit (314) present in the central zone (310) is configured to lockingly engaged the fourth flap (308) and in particular, section (308c) of the fourth flap (208) with the slit (314). The slit (314) may be curved as shown in FIG. 3 and is configured to get lockingly engaged with the section (308c) on back side of the central zone (310). In particular, when the fourth flap (308) is folded over central zone (310) and the second and third flaps specifically, section (308b) folds over the third flap (306). Further, the section (308c) is folded over the back side of the central zone (310) after passing over the third flap (306). The section (308c) may have a protrusion (308c1) within the fourth flap (308) and the protrusion (308c1) includes an end portion (308c2) having a rounded edge which may be inserted in the slit (314) on the back side of the central zone to pack the single barrier folder (300). The end portion (308c2) of the protrusion (308c1) may sit inside the slit (314) in locking engagement. The section (308a) may lie over the folded second flap (304). In particular, the fourth flap (308) is folded covering the suture and the needle which lie under the second flap (304) and the third flap (306). The curved slit (314) allows better locking engagement between the fourth flap (308) and the slit (314).

The placement of the different flaps discussed above are such that the first flap (302) and fourth flap (308) are provided on opposite sides of the plurality of openings (312a, 312b, 312c, 312d) and the first flap (302) and the fourth flap (308) are perpendicularly located with respect to the second flap (304) and the third flap (306).

The dimensions of various features provided in the single barrier folder as discussed above in FIG. 1, FIG. 2, and FIG. 3 are provided according to an exemplary embodiment of the invention. The dimensions described herein in no way limits the scope of the invention to use different and modified dimensions for various needs. The illustration of dimensions is merely to assist a person skilled in the art to perform and appreciate the invention.

The dimensions and measurements are provided in millimeter (mm) unless otherwise specified herein. The first flap may have a length of 82 mm and width of 25 mm. The first portion may be cut from three sides as discussed above and has a length of 11.65 mm and width of 12.5 mm. The cutout may have a cut depth of around 22.5 mm and width of 12.2 mm. The length of notch may be 5.8 mm and the notch forms an angle with the perforated edge between the first flap and the central zone. The angle may be 30 degrees. Further, a second cut out as discussed above may be located 31.4 mm from the right edge of the first flap as shown in FIG. 2. The second cut out may be gaussian shaped with radius of 3.0 mm and cut depth of 10.5 mm. The curved cut on the left side of the first flap may be of radius 5.5 mm along the edge of the first flap. The radius of upper left-hand side cut edge of the first flap may be 19.8 mm.

Further, the central zone has plurality of openings with horizontal separation of two openings with another two openings as shown in FIG. 1 may be 41 mm. Each opening may be 4.0 mm wide and radius of the rounded edges of the openings is 2.0 mm. The slit may have radius of 55 mm. The perforations on the boundary edge between the first flap and the central zone may be of 1.5 mm cut followed by 3 mm of gap between the perforations.

The second flap may be of 20 mm of length and 29 mm of width. The third flap may be extended and has a length of 32 mm and width of 29 mm.

The fourth flap may have dimensions with the protrusion of the fourth flap with curved edge having radius of 11.4 mm. The fourth flap may have a width of 60 mm.

Further, as discussed above, the measurements provided herein are exemplary and different dimensions and measurements pertaining to these features of the single barrier folder shown in FIG. 1, FIG. 2, and FIG. 3 are within the scope of the invention.

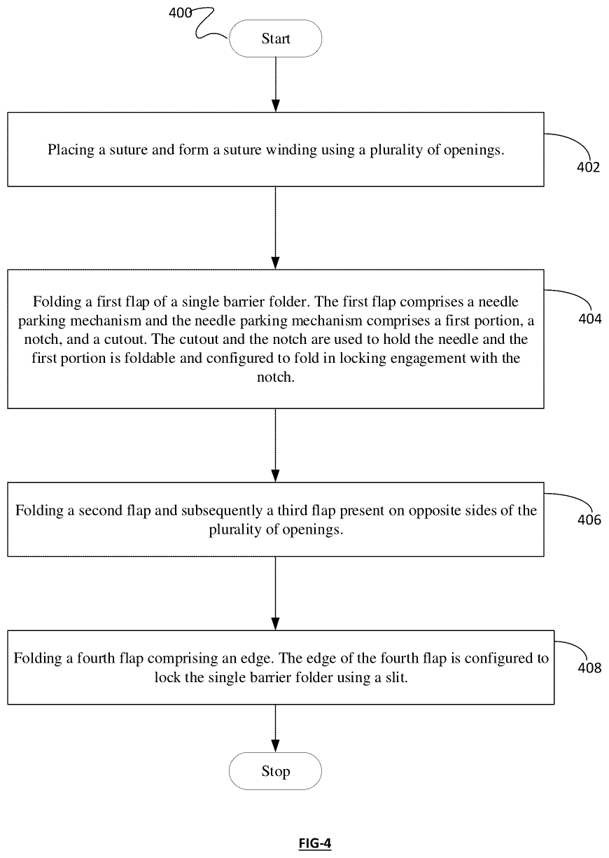

FIG. 4 represents a flowchart outlining the features of the invention in an exemplary embodiment of the invention. The method flowchart 400 describes a method of packaging the single barrier folder described above in FIG. 1 above.

At step 402, a suture is placed and a suture winding is formed using a plurality of openings. This has been explained in detail in FIG. 1.

At step 404, a first flap is folded and then a needle parking mechanism is used to park a needle. The needle parking mechanism comprises a first portion, a notch, and a cutout, the cutout and the notch are configured to hold the needle and the first portion is foldable and configured to fold in locking engagement with the notch. This has been explained in detail in FIG. 1 above.

At step 406, a second flap is folded and subsequently a third flap is folded. The second flap and the third flap are present on opposite sides of the plurality of openings. This has been explained in detail in FIG. 1 above.

At step 408, a fourth flap is folded which comprises an end portion. The end portion of the fourth flap is configured to lock a single barrier folder using a slit. This has been explained in detail in FIG. 1 above.

The method of packaging the single barrier folders described in FIG. 1, FIGS. 2 and 3 is similar to as illustrated in FIG. 4 above with respective features as discussed in FIG. 1, FIG. 2, and FIG. 3.

The present invention is applicable in any industry/field that is well known in the art such packaging of medical devices and similar components and devices. The embodiments of the invention discussed herein are exemplary and various modification and alterations to a person skilled in the art are within the scope of the invention.

When introducing elements of aspects of the invention or the examples thereof, the articles “a,” “an,” “the,” and “said” are intended to mean that there are one or more of the elements. The terms “comprising,” “including,” and “having” are intended to be inclusive and mean that there may be additional elements other than the listed elements. The term “exemplary” is intended to mean “an example of.” The phrase “one or more of the following: A, B, and C” means “at least one of A and/or at least one of B and/or at least one of C”.

Having described aspects of the invention in detail, it will be apparent that modifications and variations are possible without departing from the scope of aspects of the invention as defined in the appended claims. As various changes could be made in the above constructions, products, and methods without departing from the scope of aspects of the invention, it is intended that all matter contained in the above description and shown in the accompanying drawings shall be interpreted as illustrative and not in a limiting sense.

Although the subject matter has been described in language specific to structural features and/or acts, it is to be understood that the subject matter defined in the appended claims is not necessarily limited to the specific features or acts described above. Rather, the specific features and acts described above are disclosed as examples of implementing the claims and other equivalent features and acts are intended to be within the scope of the claims.

We claim:

1. A single barrier folder for holding a suture and a needle comprising:
a plurality of openings configured to allow placement of a suture and form a suture winding;
a first flap comprising a needle parking mechanism, wherein the needle parking mechanism comprises a first portion, a notch, and a cutout, wherein the cutout and the notch are configured to hold the needle, wherein the first portion is foldable and configured to fold in locking engagement with the notch;
a second flap and a third flap on opposite sides of the plurality of openings;
a slit configured to receive an end portion of a fourth flap to lock the single barrier folder.

2. The single barrier folder as claimed in claim 1, wherein the plurality of openings allows external rods to be inserted through the plurality of openings for forming the suture winding.

3. The single barrier folder as claimed in claim 1, wherein the plurality of openings are provided in a central zone of the single barrier folder.

4. The single barrier folder as claimed in claim 3, wherein the notch is present on a foldable perforated edge between the first flap and the central zone.

5. The single barrier folder as claimed in claim 1, wherein the first flap, second flap, and the third flap have rounded corners.

6. The single barrier folder as claimed in claim 1, wherein the surface area of the third flap is larger than surface area of the second flap.

7. The single barrier folder as claimed in claim 1, wherein the cutout is trapezoidal in shape and the notch is V-shaped.

8. The single barrier folder as claimed in claim 3, wherein the slit is provided along with the plurality of openings in the central zone, wherein the slit is curved.

9. The single barrier folder as claimed in claim 8, wherein the fourth flap comprises a protrusion which allows the end portion of the fourth flap to lock into the slit.

10. The single barrier folder as claimed in claim 9, wherein the slit is provided on a back face of the central zone, wherein the fourth flap is folded covering the suture and the needle to lock into the slit.

11. The single barrier folder as claimed in claim 1, wherein the fourth flap further comprises linear perforations to tear off a portion of fourth flap.

12. The single barrier folder as claimed in claim 1, wherein the notch and cutout are configured to hold the needle which is curved.

13. The single barrier folder as claimed in claim 1, wherein the first flap and fourth flap are provided on opposite sides of the plurality of openings, wherein the first flap and fourth flap are perpendicularly located with respect to the second flap and the third flap.

14. A method comprising:
placing a suture and forming a suture winding using a plurality of openings;
folding a first flap, wherein the first flap comprises a needle parking mechanism, wherein the needle parking mechanism comprises a first portion, a notch, and a cutout, wherein the cutout and the notch are configured to hold the needle, wherein the first portion is foldable and configured to fold in locking engagement with the notch;
folding a second flap and subsequently a third flap, wherein the second flap and the third flap are present on opposite sides of the plurality of openings;
folding a fourth flap comprising an end portion, wherein the end portion of the fourth flap is configured to lock a single barrier folder using a slit.

15. The method as claimed in claim 14, wherein the plurality of openings allows external rods to be inserted through the plurality of openings for forming the suture winding.

16. The method as claimed in claim 14, wherein the plurality of openings are provided in a central zone of the single barrier folder.

17. The method as claimed in claim 16, wherein the notch is present on a foldable perforated edge between the first flap and the central zone.

18. The method as claimed in claim 14, wherein the first flap, second flap and the third flap have rounded corners.

19. The method as claimed in claim 14, wherein the surface area of the third flap is larger than the second flap.

20. The method as claimed in claim 14, wherein the cutout is trapezoidal in shape and the notch is V-shaped.

21. The method as claimed in claim 16, wherein the slit is provided along with the plurality of openings in the central zone, wherein the slit is curved.

22. The method as claimed in claim 21, wherein the fourth flap comprises a protrusion which allows the end portion of the fourth flap to lock into the slit.

23. The method as claimed in claim 22, wherein the slit is provided on a back face of the central zone, wherein the fourth flap is folded covering the suture and the needle to lock into the slit.

24. The method as claimed in claim 14, wherein the fourth flap further comprises linear perforations to tear off a portion of fourth flap.

25. The method as claimed in claim 14, wherein the notch and cutout are configured to hold the needle which is curved.

26. The method as claimed in claim 14, wherein the first flap and fourth flap are provided on opposite sides of the plurality of openings, wherein the first flap and fourth flap are perpendicularly located with respect to the second flap and the third flap.

27. A single barrier folder for holding a suture and a needle comprising:
a plurality of openings configured to allow placement of a suture and form a suture winding;
a first flap comprising a needle parking mechanism, wherein the needle parking mechanism comprises a first portion, a notch, a first cutout and a second cutout, wherein the first cutout and the second cutout are configured to hold the needle, wherein the first portion is foldable and configured to fold in locking engagement with the notch; and
a second flap and a third flap on opposite sides of the plurality of openings;
a slit configured to receive an end portion of a fourth flap to lock the single barrier folder, wherein the slit is curved.

28. A single barrier folder for holding a suture and a needle comprising:
a plurality of openings configured to allow placement of a suture and form a suture winding;
a first flap comprising a foam section for holding the needle;
a second flap and a third flap on opposite sides of the plurality of openings;
a slit configured to receive an end portion of a fourth flap to lock the single barrier folder, wherein the slit is curved.
